(19) United States
(12) Patent Application Publication
(10) Pub. No.: US 2020/0192401 A1
(43) Pub. Date: Jun. 18, 2020

(54) METHOD AND DEVICE FOR DETERMINING A HIGHLY-PRECISE POSITION AND FOR OPERATING AN AUTOMATED VEHICLE

(71) Applicant: Robert Bosch GmbH, Stuttgart (DE)

(72) Inventors: Daniel Zaum, Sarstedt (DE); Holger Mielenz, Ostfildern (DE); Jan Rohde, Stuttgart (DE)

(73) Assignee: Robert Bosch GmbH, Stuttgart (DE)

(21) Appl. No.: 16/638,910

(22) PCT Filed: Jul. 12, 2018
(86) PCT No.: PCT/EP2018/068884

§ 371 Date: Feb. 13, 2020

(30) Foreign Application Priority Data
Aug. 23, 2017 (DE) 10 2017 214 729.6

(51) Int. Cl. G05D 1/02; G01C 21/28; G01S 13/931; G01N 33/00; G05D 1/00

(52) U.S. Cl. CPC G05D 1/0274; G01C 21/28; G01S 13/931; G01N 33/0036; G01S 5/00; G05D 1/0276; G01S 2013/9322; G01S 2013/9323; G01S 2013/9324; G05D 1/0088

(57) ABSTRACT

A method and a device for determining a highly-precise position and for operating an automated vehicle, including receiving map data values from an external server, which represent a map, the map including weather-specific surroundings features, determining a weather-specific surroundings condition, detecting surroundings data values, the surroundings data values representing the surroundings of the automated vehicle, the surroundings including dynamic surroundings features, determining the highly-precise position based on a comparison between the weather-specific surroundings features and the dynamic surroundings features depending on the weather-specific surroundings condition, and operating the automated vehicle, depending on the highly-precise position.

### FIELD

The present invention relates to a method and a device for determining a highly-precise position and for operating an automated vehicle, including a step of receiving map data values from an external server, a step of determining a weather-specific surroundings condition, a step of detecting the surroundings data values, a step of determining the highly-precise position, and a step of operating the automated vehicle, depending on the highly-precise position.

### SUMMARY

An example method according to the present invention for determining a highly-precise position and for operating an automated vehicle includes a step of receiving map data values from an external server, which represent a map, the map including weather-specific surroundings conditions, a step of determining a weather-specific surroundings condition, and a step of detecting surroundings data values, the surroundings data values representing the surroundings of the automated vehicle, the surroundings including dynamic surroundings features. The method according to the present invention further includes a step of determining the highly-precise position, based on a comparison between the weather-specific surroundings features and the dynamic surroundings features, depending on the weather-specific surroundings condition, and a step of operating the automated vehicle, depending on the highly-precise position. An automated vehicle is understood to be a semi, highly, or fully automated vehicle.

Operating the automated vehicle is understood to mean that the automated vehicle is operated in a semi, highly, or fully automated manner. The operation includes, for example, the determination of a trajectory for the automated vehicle and/or the drive along the trajectory with the aid of an automated transverse and/or longitudinal controller and/or carrying out safety-relevant driving functions, etc.

A highly-precise position is understood to be a position which is precise within a predefined coordinate system, for example, GNSS coordinates, in such a way that this position does not exceed a maximum permissible lack of definition. The maximum lack of definition may thereby depend, for example, on the surroundings of the automated vehicle. Furthermore, the maximum lack of definition may depend, for example, on whether the automated vehicle is operated in a semi, highly, or fully automated manner. Basically, the maximum lack of definition is so low that a safe operation of the automated vehicle is ensured. For fully automated operation of the automated vehicle, the maximum lack of definition lies, for example, in the range of approximately 10 centimeters.

The surroundings of the automated vehicle are understood, for example, to be an area which may be detected with the aid of a surroundings sensor system of the vehicle.

A map is understood to be, for example, a digital map, which is configured, for example, in connection with a navigation system and/or a control unit of the automated vehicle and/or in connection with a smartphone, which is connected to the automated vehicle or is included in the same, to localize the automated vehicle and/or to carry out a function, depending on the localization, etc.

The example method according to the present invention solves the problem in an advantageous way, in that a safe and reliable operation of an automated vehicle depends in many cases on the knowledge of a highly-precise position of the automated vehicle. In general, multiple methods for determining the highly-precise position are available, some of the methods functioning more reliably than others—for example, depending on certain surroundings influences.

The method described here supports the determination of the highly-precise position, in particular during poor weather conditions. Particularly during rain and/or snow fall, conventional localization systems may lead to a considerable limitation of availability and/or precision of the localization, which over all leads to a limitation in operating the automated vehicle.

An evaluation of the comparison is preferably carried out according to predefined criteria and is transmitted to the external server depending on the evaluation of at least one of the dynamic surroundings features.

The predefined criteria establish, for example, whether the at least one of the dynamic surroundings features have been detected with a highly-precise position or not, this at least one dynamic surroundings feature being then only transmitted if the highly-precise position is known.

This yields the advantage that the automated vehicle itself contributes, for example, to an improvement and/or updating of the map, which may then be provided to other (automated) vehicles.

The weather-specific surroundings features were preferably previously detected by at least one other vehicle and transmitted to the external server.

This yields the advantage that the map, received from the external server, also includes up-to-the-minute weather-specific surroundings features, whereby the highly-precise position may be determined, for example, more reliably and/or more precisely.

The weather-specific surroundings features and/or the dynamic surroundings features preferably encompass light reflections and/or traffic lanes of the at least one additional vehicle.

Light reflections are to be understood, for example, as headlights and/or lights from street lights, neon signs, display windows, traffic signs, etc., which are reflected on the wet and/or snow-covered roadway. Traffic lanes are understood, for example, to be tracks which emerge due to the wet and/or snow-covered roadway.

Furthermore, a weather-specific surroundings feature and/or a dynamic surroundings feature is understood to be, for example, an area at which water collects, etc., during and/or after precipitation, for example, due to uneven road surfaces.

This yields the advantage that poor weather conditions themselves lead to additional (weather-specific, dynamic) surroundings features, which are used to determine the highly-precise position, whereas other surroundings features may not be used, particularly during poor weather conditions.

The example device according to the present invention for determining a highly-precise position and for operating an automated vehicle includes first means for receiving map data values from an external server, which represent a map, the map including weather-specific surroundings feature, second means for determining weather-specific surroundings conditions, and third means for detecting surroundings data values, the surroundings data values representing the surroundings of the automated vehicle, the surroundings including dynamic surroundings features. The device according to the present invention further includes fourth means for determining the highly-precise position, based on a comparison between the weather-specific surroundings features and the dynamic surroundings features, depending on the weather-specific surroundings condition, and fifth means for operating the automated vehicle depending on the highly-precise position.

The first means and/or the second means and/or the third means and/or the fourth means and/or the fifth means are preferably configured to carry out a method according to at least one of the method claims.

Advantageous refinements of the present invention are described herein.

### BRIEF DESCRIPTION OF THE DRAWINGS

Exemplary embodiments of the present invention are depicted in the figures and are described in greater detail below.

FIG. 1 shows purely by example a first exemplary embodiment of the device according to the present invention.

FIG. 2 shows purely by example a second exemplary embodiment of the device according to the present invention.

FIG. 3 shows purely by example an exemplary embodiment of the method according to the present invention in the form of a flow chart.

### DETAILED DESCRIPTION OF EXAMPLE EMBODIMENT

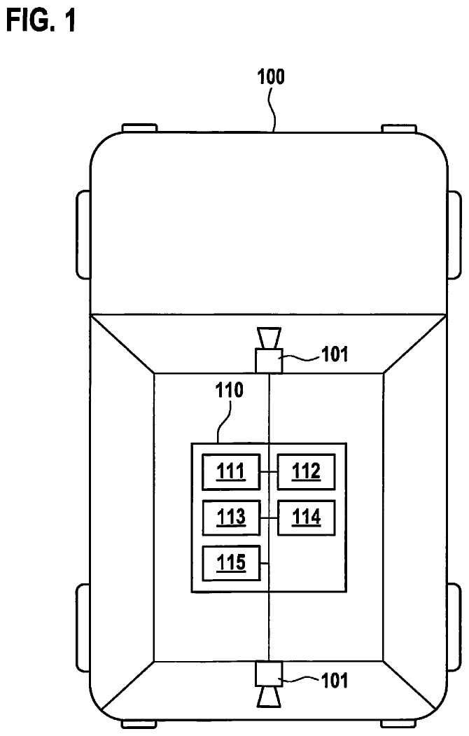

FIG. 1 shows an automated vehicle 100, which includes device 110 according to the present invention for determining 340 a highly-precise position 150 and for operating 350 automated vehicle 100.

Device 110 includes first means 111 for receiving 310 map data values from an external server 210, which represent a map, the map including weather-specific surroundings features 220, second means 112 for determining 320 a weather-specific surroundings condition, and third means 113 for detecting 330 surroundings data values, the surroundings data values representing surroundings 200 of automated vehicle 100, the surroundings including dynamic surroundings features 230. Device 110 further includes fourth means 114 for determining 340 highly-precise position 150, based on a comparison between weather-specific surroundings features 220 and dynamic surroundings features 230, depending on the weather-specific surroundings condition, and fifth means 115 for operating 350 automated vehicle 100, depending on highly-precise position 150.

First means 111 for receiving 310 map data values from an external server 210 is configured, for example, as a transmitting and/or receiving unit. In another specific embodiment, first means 111 is configured in such a way that it is already connected to a transmitting and/or receiving unit included in the vehicle.

Second means 112 for determining 320 a weather-specific surroundings condition is configured, for example, as a transmitting and/or receiving unit, which requests the weather-specific surroundings condition, for example, from a weather station and/or another external server. In one specific embodiment, the transmitting and/or receiving unit is/are identical to the transmitting and/or receiving unit of first means 111.

In another specific embodiment, second means 112 is configured in such a way that the weather-specific surroundings condition is detected with the aid of a surroundings sensor system 101, which is included in automated vehicle 100. For this purpose, second means 112 additionally includes, for example, a processing unit (processor, working memory, hard disk, software), which is configured to correspondingly evaluate surroundings data, which are detected with the aid of surroundings sensor system 101—for example, in the form of an image from a video sensor and/or in the form of humidity values from a humidity sensor.

Third means 113 for detecting 330 surroundings data values is designed, for example, in such a way that they have an inherent surroundings sensor system or is connected to surroundings sensor system 101 already included in automated vehicle 100. Furthermore, third means includes, for example, a processing unit (processor, working memory, hard disk, software), which processes and evaluates the surroundings data values.

Surroundings sensor system 101 is understood to be, for example, at least one video and/or at least one radar and/or at least one LIDAR, and/or at least one ultrasound and/or at least one additional sensor, which is/are configured to detect surroundings 200 of automated vehicle 100 in the form of surroundings data values.

Fourth means 114 for determining 340 highly-precise position 150, based on a comparison between weather-specific surroundings features 220 and dynamic surroundings features 230, depending on the weather-specific surroundings condition, is configured, for example, as a control unit and/or processing unit. It includes, for example, a processor, a working memory, and a hard disk, as well as a suitable software for determining 340 a highly-precise position 150 of automated vehicle 100.

Fifth means 115 for operating 350 automated vehicle 100, depending on highly-precise position 150, is configured, for example, as a control unit.

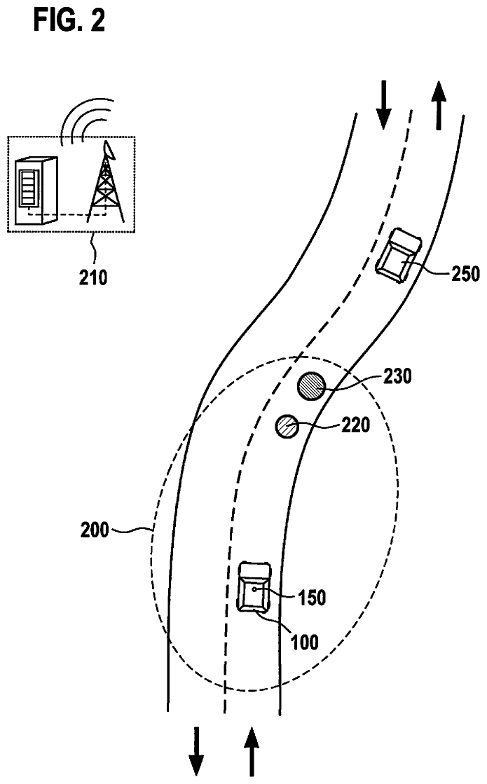

FIG. 2 shows a schematic depiction of one exemplary embodiment of method 300 according to the present invention. An automated vehicle 100 thereby drives in an automated manner on a road.

The automated vehicle receives map data values from an external server 210 with the aid of first means 111, the map data values representing a map, the map including weather-specific surroundings features 220. In one specific embodiment, the map data values are received, for example, at regular time intervals and/or location intervals, depending on the (not highly-precise) position of automated vehicle 100. In another specific embodiment, automated vehicle 100 requests the map data values, for example, if no up-dated map is present and/or a determination 340 of a highly-precise position 150 is not possible. In another specific embodiment, the map data values are transmitted from external server 210 if, for example, an update of the map has been carried out.

Automated vehicle 100 further determines a weather-specific surroundings condition with the aid of second means 112. In one specific embodiment, this step is carried out in that the weather-specific surroundings condition is transmitted, together with the map data values, from external server 210 and are received with the aid of first means 111. In another specific embodiment, the weather-specific surroundings condition is determined independently from the received map data values—for example, with the aid of surroundings sensor system 101 of automated vehicle 100.

Automated vehicle 100 further detects surroundings data values, the surroundings data values representing the surroundings 200 of automated vehicle 100, surroundings 200 including dynamic surroundings features 230.

In one specific embodiment, the dynamic surroundings feature corresponds, for example, to a traffic lane of at least one other vehicle 250, which, for example, previously transmits its own highly-precise position—in regular intervals—to external server 210. External server 210 in turn transmits the map data values, the map now including the expected traffic lane of the at least one additional vehicle 250 as weather-specific surroundings feature 220—the track not being visible on a dry roadway.

This track is now detected with the aid of third means 113 of automated vehicle 100 as dynamic surroundings feature 230.

Subsequently, highly-precise position 150 of automated vehicle 100 is determined, based on a comparison between weather-specific surroundings features 220 and dynamic surroundings features 230 (here, for example, the track of at least one additional vehicle 250 on the wet roadway), depending on the weather-specific surroundings condition. The weather-specific surroundings condition is thereby used, for example, to determine the actual highly-precise position 150, since due to this state appropriate parameters are used based. In another specific embodiment, the weather-specific surroundings condition decides, for example, whether the weather-specific surroundings feature is suited for determining 340 highly-precise position 150. For example, the track may be better suited during light rain for being detected with the aid of third means 113, than during very heavy rain, since the track is hardly to be recognized due to increasing water volumes.

Highly-precise position 150 is determined, for example, in that dynamic surroundings feature 230 is detected and a relative position of automated vehicle 100 to this is determined. This is carried out, for example, with the aid of a directional vector and a distance between dynamic surroundings feature 230 and automated vehicle 100. Since the likewise highly-precise position of weather-specific surroundings feature 220 is recorded in the map data values, highly-precise position 150 of automated vehicle 100 is determined, starting from this position and the relative position, for example, with the aid of vector addition.

In another specific embodiment, a light reflection is used, for example, as weather-specific feature 220, which may be detected as dynamic surroundings feature 230 with the aid of surroundings sensor system 101, as long as, for example, the road, on which automated vehicle 100 is located, has a wet road surface.

In one specific embodiment, a dynamic surroundings feature 230, which is not included in the map, is detected by automated vehicle 100 and transmitted to external server 210.

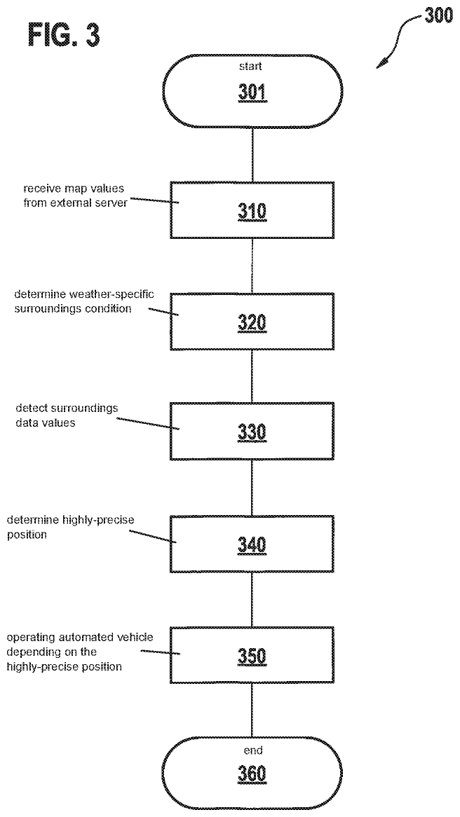

FIG. 3 shows an exemplary embodiment of a method 300 for determining 340 a highly-precise position 150 and for operating 350 an automated vehicle 100.

Method 300 starts in step 301.

In step 310, map data values, which represent a map, the map including weather-specific surroundings features 220, are received from an external server 210.

In step 320, a weather-specific surroundings condition is determined.

In step 330, surroundings data values are detected, the surroundings data values representing the surroundings 200 of automated vehicle 100, the surroundings 200 including dynamic surroundings features 230.

In step 340, highly-precise position 150 is determined, based on a comparison between weather-specific surroundings features 220 and dynamic surroundings features 230, depending on the weather-specific surroundings condition.

In step 350, automated vehicle 100 is operated depending on highly-precise position 150.

Method 300 ends in step 360.

1-6 (canceled)

7. A method for determining a highly-precise position and for operating an automated vehicle, the method comprising the following steps:
receiving map data values from an external server, the map data representing a map, the map including weather-specific surroundings features;
determining a weather-specific surroundings condition;
detecting surroundings data values, the surroundings data values representing surroundings of the automated vehicle, the surroundings including dynamic surroundings features;
determining the highly-precise position, based on a comparison between the weather-specific surroundings features and the dynamic surroundings features, depending on the weather-specific surroundings condition; and
operating the automated vehicle, depending on the highly-precise position.

8. The method as recited in claim 7, wherein an evaluation of the comparison is carried out depending on predefined criteria, and depending on the evaluation, at least one of the dynamic surroundings features is transmitted to the external server.

9. The method as recited in claim 7, wherein the weather-specific surroundings features were previously detected by at least one additional vehicle and were transmitted to the external server.

10. The method as recited in claim 9, wherein the weather-specific surroundings features and/or the dynamic surroundings features include: (i) light reflections, and/or (ii) tracks of the at least one additional vehicle.

11. A device for determining a highly-precise position and for operating an automated vehicle, the device comprising:
a first device configured to receive map data values from an external server, the map data values representing a map, the map including weather-specific surroundings features;
a second device configured to determine a weather-specific surroundings condition;
a third device configured to detect surroundings data values, the surroundings data values representing surroundings of the automated vehicle, the surroundings including dynamic surroundings features;
a fourth device configured to determine the highly-precise position, based on a comparison between the weather-specific surroundings features and the dynamic surroundings features, depending on the weather-specific surroundings condition; and
a fifth device configured to operate the automated vehicle, depending on the highly-precise position.

12. The device as recited in claim 11, wherein the first device and/or the second device and/or the third device and/or the fourth device and/or the fifth device, is configured to carry out a method comprising:
receiving the map data values from the external server;
determining the weather-specific surroundings condition;
detecting the surroundings data values;
determining the highly-precise position, based on the comparison between the weather-specific surroundings features and the dynamic surroundings features, depending on the weather-specific surroundings condition; and
operating the automated vehicle, depending on the highly-precise position.

13. The device as recited in claim 11, wherein the first device includes a receiver, wherein the second device, the third device, and the fourth device include a processor, and wherein the fourth device includes a control unit.
